(19) United States
(12) Patent Application Publication
(10) Pub. No.: US 2024/0239768 A1
(43) Pub. Date: Jul. 18, 2024

(54) METHOD FOR PREPARING XANTHINE OXIDASE INHIBITOR

(71) Applicant: LG CHEM, LTD., Seoul (KR)

(72) Inventors: In Ae RYU, Seoul (KR); Ju Young LEE, Cheongju-si (KR); Joo Yong YOON, Seoul (KR); Seok Ju LEE, Seoul (KR); Ah Byeol PARK, Seoul (KR); Ki Dae KIM, Seoul (KR); Hui Rak JEONG, Seoul (KR)

(73) Assignee: LG CHEM, LTD., Seoul (KR)

(21) Appl. No.: 18/557,276

(22) PCT Filed: Apr. 26, 2022
(86) PCT No.: PCT/KR2022/005926

§ 371 Date: Oct. 25, 2023

(30) Foreign Application Priority Data
Apr. 27, 2021 (KR) 10-2021-0054151

(51) Int. Cl. C07D 403/04

(52) U.S. Cl. CPC C07D 403/04

(57) ABSTRACT

The present invention relates to a novel preparation method that can be advantageously utilized for the synthesis of a xanthine oxidase inhibitor of Chemical Formula 1.

### TECHNICAL FIELD

The present invention relates to a novel method for preparing a xanthine oxidase inhibitor, more specifically to a novel preparation method that can be utilized to more efficiently synthesize a xanthine oxidase inhibitor of Chemical Formula 1 using a compound of Chemical Formula 2 as a starting material through a simple process:

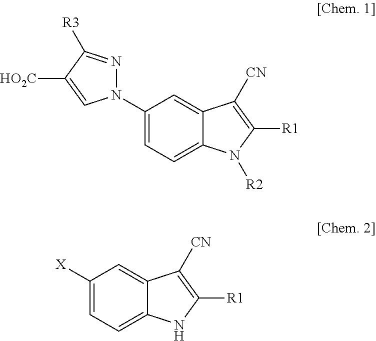

wherein

R1 is hydrogen, halogen, C₁-C₇ alkyl, C₁-C₇ alkoxy-C₁-C₇ alkyl or phenyl;

R2 is hydrogen; C₁-C₇ alkyl unsubstituted or substituted with a substituent selected from halogen, C₃-C₇ cycloalkyl or O—R6, wherein R6 represents C₁-C₄ alkyl; C₃-C₇ cycloalkyl; or

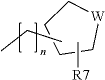

(wherein W represents O or S, R7 represents hydrogen or C₁-C₄ alkyl, and n is an integer 0 to 3);

R3 is hydrogen, halogen or C₁-C₇ alkyl; and

X is F, Cl, Br or I.

### BACKGROUND ART

Xanthine oxidase is known as an enzyme that converts hypoxanthine to xanthine and the formed xanthine to uric acid. Since uricase, which exists in most mammals, does not exist in humans and chimpanzees, a substance called uric acid is known to be the final product of purine metabolism (S. P. Bruce, Ann. Pharm., 2006, 40, 2187-2194). Uric acid contained in the blood at high concentrations causes various diseases, a representative example of which is gout.

As mentioned above, gout is a disease caused by high levels of uric acid in the body, and refers to a condition in which uric acid crystals accumulate in joint cartilage, ligaments, and surrounding tissues, causing severe inflammation and pain. Gout is a type of inflammatory joint disease, the incidence of which has been steadily increasing over the past 40 years (N. L. Edwards, Arthritis & Rheumatism, 2008, 58, 2587-2590).

Looking at the number of patients with gout in Western countries from the 1960s to the mid-1990s, there is a surprising increase of about 200% to 300%, and patients with gout are mainly men. Obesity, aging, decreased kidney function, high blood pressure, and the like are considered to be the causes of the rate of increase in patients with gout. The incidence of gout is about 1.4/1000 people, but also vary depending on the level of uric acid. In other words, the incidence of gout in patients having a uric acid level in the blood of 7.0 mg/dl or more is 0.5%, but the incidence of gout in patients having a uric acid level in the blood of 9.0 mg/dl or more is 5.5% (G. Nuki, Medicine, 2006, 34, 417-423). Considering the incidence, it can be seen that uric acid concentration in the blood is an important factor causing gout. Additionally, dietary habits, alcohol, lipids, obesity, and the like may also act as important factors causing gout. These days, studies on the correlation between uric acid and heart failure, high blood pressure, diabetes, kidney disease, and cardiovascular disease are being actively carried out by a number of researchers, and the importance of uric acid management is increasing (D. I. Feig et al., N. Eng. J. Med, 2008, 23, 1811-1821). In addition, allopurinol, a xanthine oxidase inhibitor, is known to be effective in ulcerative colitis (Aliment. Pharmacol. Ther. 2000, 14, 1159-1162; WO 2007/043457).

Until febuxostat was approved as an arthrifuge in the United States in 2009 (Brain Tomlinson, Current opin. invest. drugs, 2005, 6, 1168-1178), allopurinol was the only drug used to treat gout for the past 40 years. Allopurinol is known as a non-specific inhibitor of various enzymes involved in purine and pyrimidine metabolism, and has a Ki of 700 nM against xanthine oxidase (Y. Takano et al., Life Sciences, 2005, 76, 1835-1847). Allopurinol is directly oxidized by xanthine oxidase and converted to oxypurinol, and this metabolite is known to act as a greatly strong inhibitor of xanthine oxidase.

However, allopurinol is known to cause gastrointestinal side effects and skin rashes, and to have poor compliance when taken for a long period of time. In particular, among patients taking allopurinol, it has been reported that an unpredictable fatal side effect of Stevens-Johnson syndrome occurs at a low ratio (Felix Arellano et al, Ann. Pharm., 1993, 27, 337-43). This side effect is known to be a serious side effect that causes cell necrosis in the skin and mucous membranes of the mouth, leading to death in about 25% of cases if not treated appropriately.

Accordingly, various studies have been conducted to develop new xanthine oxidase inhibitors, and Korean Patent Publication No. 10-2011-0037883 discloses a novel compound of the following Chemical Formula 1, which is effective as a xanthine oxidase inhibitor:

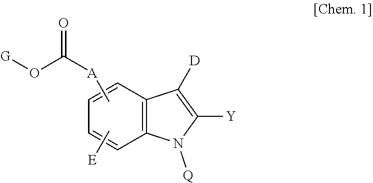

in Chemical Formula 1.

A is selected from the following substituents A-i, A-ii, A-iii, A-iv, A-v, A-vi, A-vii and A-viii,

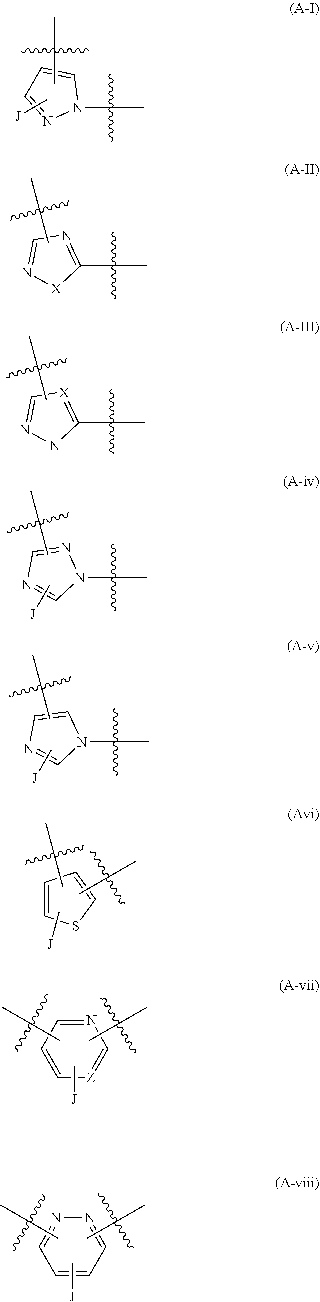

wherein

J represents hydrogen, halogen, or C₁-C₆-alkyl unsubstituted or substituted with halogen,

X is O or S, and

Z is Cor N.

E represents hydrogen, halogen, cyano, nitro, substituted or unsubstituted C₁-C₆-alkyl, or substituted or unsubstituted C₁-C₆-alkoxy,

D represents hydrogen, halogen, cyano, nitro, C₁-C₆-alkyl unsubstituted or substituted with halogen, —CHO, or —CH═N—OH,

Q is selected from the following substituents Q-i, Q-ii, and Q-iii-1 to Q-iii-9

(Q-i) hydrogen;

(Q-ii) substituted or unsubstituted linear, branched or cyclic, saturated or unsaturated alkyl;

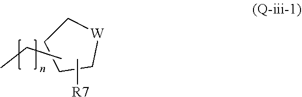

(wherein W represents O or S, R7 represents hydrogen or substituted or unsubstituted lower alkyl, and n is an integer 0 to 3);

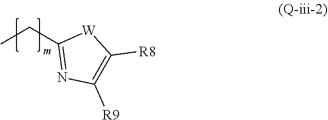

(wherein W represents O or S, R8 and R9 each independently represent hydrogen or lower alkyl, and m is an integer 1 to 3);

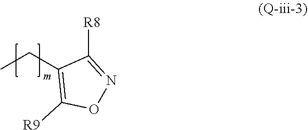

(wherein R8 and R9 each independently represent hydrogen or lower alkyl, and m is an integer 1 to 3);

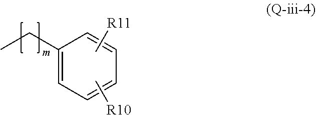

(wherein, R10 and R11 each independently represent hydrogen, halogen, lower alkoxy, or lower alkyl, and m is an integer 1 to 3);

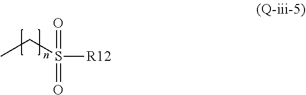

(wherein R12 represents substituted or unsubstituted lower alkyl or aromatic, and n is an integer 0 to 3);

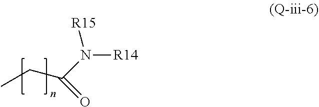

(wherein, R13 and R14 each independently represent substituted or unsubstituted lower alkyl, or may form a 3- to 7-membered heterocycle containing N, and n is an integer 0 to 3);

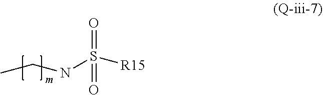

(wherein R15 represents substituted or unsubstituted lower alkyl, and m is an integer 1 to 3);

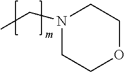

(wherein m is an integer 1 to 3); and

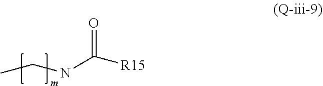

(wherein R15 represents substituted or unsubstituted lower alkyl, and m is an integer 1 to 3),

Y represents hydrogen, halogen, substituted or unsubstituted linear, branched or cyclic saturated or unsaturated alkyl, substituted or unsubstituted C₁-C₆-alkoxy, substituted or unsubstituted aromatic, or heteroaromatic, and

G represents hydrogen or substituted or unsubstituted linear, branched or cyclic, saturated or unsaturated alkyl.

In a specific example of the document, the preparation of 1-(3-cyano-1-isopropyl-indol-5-yl)pyrazole-4-carboxylic acid according to the following Scheme 1 is disclosed.

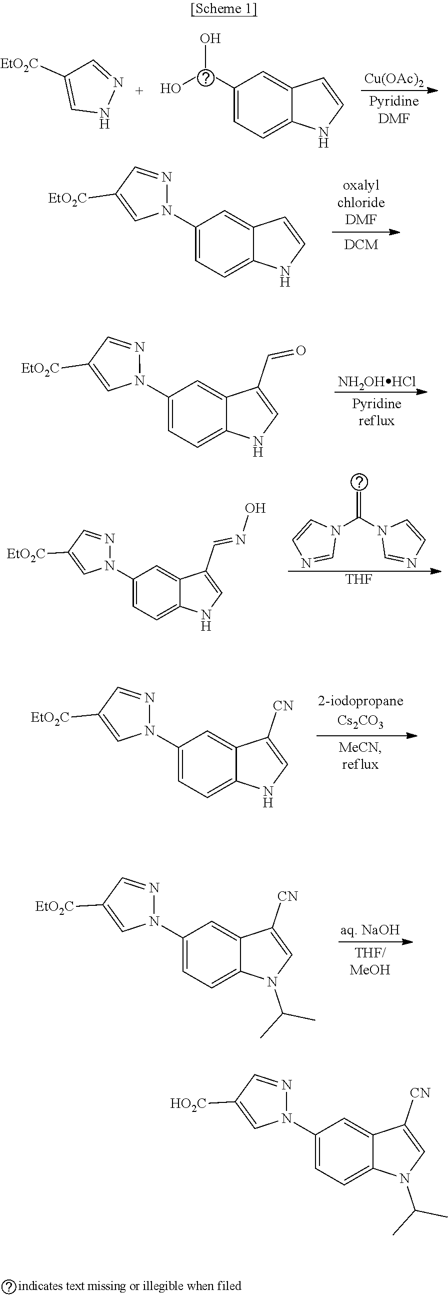

Scheme 1 is described in more detail as follows.

(1) 1H-pyrazole-4-carboxylic acid ethyl ester and 1H-indol-5-ylboronic acid are dissolved in N,N-dimethylformamide (DMF), copper(II) acetate and pyridine are added, the mixture is stirred at room temperature for 3 days, then the solvent is distilled off under reduced pressure, and separation is performed by column chromatography to prepare 1-(1H-indol-5-yl)pyrazole-4-carboxylic acid ethyl ester.

(2) 1-(1H-indol-5-yl)pyrazole-4-carboxylic acid ethyl ester is added to the reaction solution of oxalyl chloride and N,N-dimethylformamide and then reacted, and the organic layer is dried over anhydrous magnesium sulfate and concentrated under reduced pressure to prepare 1-(3-formyl-1H-indol-5-yl)pyrazole-4-carboxylic acid ethyl ester.

(3) 1-(3-formyl-1H-indol-5-yl)pyrazole-4-carboxylic acid ethyl ester is dissolved in pyridine, hydroxyammonium chloride is added, and the mixture is heated and stirred under reflux. When the reaction is completed, the reaction mixture is concentrated under reduced pressure and filtered through silica gel to prepare 1-[3-[(E,Z)-hydroxyiminomethyl]-1H-indol-5-yl]pyrazole-4-carboxylic acid ethyl ester.

(4) 1-[3-[(E,Z)-hydroxyiminomethyl]-1H-indol-5-yl]pyrazole-4-carboxylic acid ethyl ester is dissolved in anhydrous tetrahydrofuran, di(imidazol-1-yl)methanethione is added, the reaction is conducted at room temperature while stirring is performed, the reaction mixture is concentrated under reduced pressure, and the produced solid compound is separated by column chromatography to prepare 1-(3-cyano-1H-indol-5-yl)pyrazole-4-carboxylic acid ethyl ester.

(5) 1-(3-cyano-1H-indol-5-yl)pyrazole-4-carboxylic acid ethyl ester is dissolved in acetonitrile, then cesium carbonate and 2-iodopropane are added, and the mixture is heated and stirred under reflux. When the reaction is completed, the reaction mixture is concentrated under reduced pressure, and the produced solid compound is separated by column chromatography to prepare 1-(3-cyano-1-isopropyl-indol-5-yl)pyrazole-carboxylic acid ethyl ester.

(6) 1-(3-cyano-1-isopropyl-indol-5-yl)pyrazole-4-carboxylic acid ethyl ester is added to tetrahydrofuran, methanol and 6 N sodium hydroxide solution, the reaction is conducted at room temperature, then the organic solvent is removed under reduced pressure, the remaining aqueous solution layer is washed with ethyl acetate, concentrated hydrochloric acid is added to acidify the aqueous solution to pH 1, and the precipitated solid compound is filtered, washed with distilled water, and dried to prepare 1-(3-cyano-1-isopropyl-indol-5-yl)pyrazole-4-carboxylic acid.

However, the method includes several synthesis steps, and may not be preferable for mass synthesis of xanthine oxidase inhibitors at high yields.

### SUMMARY OF INVENTION

### Technical Problem

Accordingly, the technical object of the present invention is to provide a method suitable for more efficient mass production of a compound of Chemical Formula 1, which is an excellent xanthine oxidase inhibitor.

### Solution to Problem

In order to achieve the object, the present invention provides a method for preparing a xanthine oxidase inhibitor of the following Chemical Formula 1, which includes the following steps:
i) preparing a compound of Chemical Formula 3 by introducing an R2 substituent into a compound of Chemical Formula 2,
ii) reacting the compound of Chemical Formula 3 with a compound of the following Chemical Formula 4 to prepare a compound of Chemical Formula 5, and
iii) preparing a compound of Chemical Formula 1 by removing an R4 substituent from the compound of Chemical Formula 5:

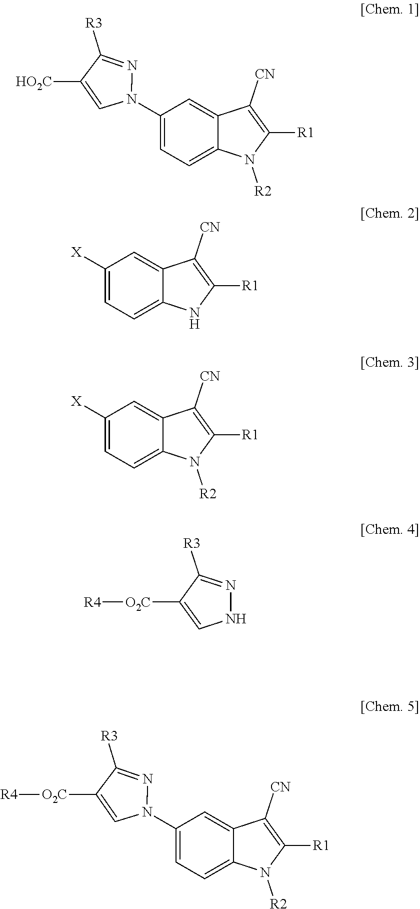

wherein,

R1 is hydrogen, halogen, C₁-C₇ alkyl, C₁-C₇ alkoxy-C₁-C₇ alkyl or phenyl;

R2 is hydrogen; C₁-C₇ alkyl unsubstituted or substituted with a substituent selected from halogen, C₃-C₇ cycloalkyl or O—R6, where R6 represents C₁-C₄ alkyl; C₃-C₇ cycloalkyl; or

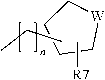

(where W represents O or S, R7 represents hydrogen or C₁-C₄ alkyl, and n is an integer 0 to 3);

R3 is hydrogen, halogen or C₁-C₇ alkyl;

R4 is C₁-C₇ alkyl or C₃-C₇ cycloalkyl; and

X is F, Cl, Br or I.

Hereinafter, the present invention will be described in detail.

In the method for preparing a xanthine oxidase inhibitor of Chemical Formula 1 according to the present invention, first, a compound of Chemical Formula 2 is reacted with an appropriate substituent in an organic solvent to prepare a compound of Chemical Formula 3.

In an embodiment according to the present invention, R2 is unsubstituted C₁-C₇ alkyl, for example, R2 is isopropyl, and the compound of Chemical Formula 3 can be prepared by reaction of the compound of Chemical Formula 2 with 2-iodopropane and CS₂CO₃.

In the method for preparing a xanthine oxidase inhibitor of Chemical Formula 1 according to the present invention, a compound of Chemical Formula 5 is prepared by reacting the compound of Chemical Formula 3 with a compound of Chemical Formula 4 in the presence of a copper catalyst, a base, and a ligand in an organic solvent.

In an embodiment according to the present invention, as the organic solvent in the step, for example, one or more selected from toluene, xylene, dimethylformamide (DMF), or dimethyl sulfoxide (DMSO) may be used.

In another embodiment according to the present invention, as the copper catalyst in the step, for example, one or more selected from CuI, Cu(OAc)₂, Cu, Cu₂O or CuO may be used.

In an embodiment according to the present invention, as the base in the step, for example, one or more selected from potassium carbonate (K₂CO₃), cesium carbonate (Cs₂CO₃), potassium phosphate tribasic (K₃PO₄), triethylamine (Et₃N) or sodium tert-butoxide (NaOtBu) may be used.

In an embodiment according to the present invention, as the ligand in the step, for example, one or more selected from 1,2-cyclohexanediamine, N,N′-dimethyl-1,2-cyclohexanediamine, N,N′-dimethylethylenediamine, 1,10-phenanthroline, proline, an oxime ligand or a tetradentate ligand may be used.

In the method for preparing a xanthine oxidase inhibitor of Chemical Formula 1 according to the present invention, a compound of Chemical Formula 1 can be prepared by removing an R4 substituent from the compound of Chemical Formula 5.

In an embodiment according to the present invention, R4 is C₁-C₇ alkyl, and for example, the xanthine oxidase inhibitor of Chemical Formula 1 can be prepared by hydrolyzing an ester with a base.

In an embodiment according to the present invention, the base in the step may be one or more selected from sodium hydroxide (NaOH), lithium hydroxide (LiOH), calcium hydroxide (Ca(OH)₂), or potassium hydroxide (KOH).

### Advantageous Effects of Invention

In the preparation method of the present invention, as a compound of Chemical Formula 3 is prepared using a compound of Chemical Formula 2 into which a nitrile group is introduced as a starting material, and then a compound of Chemical Formula 5 is prepared through a C—N coupling reaction of the compound of Chemical Formula 3 with a compound of Chemical Formula 4, a xanthine oxidase inhibitor of Chemical Formula 1 can be prepared at a high yield under mild conditions through a simpler process.

### DESCRIPTION OF EMBODIMENTS

Hereinafter, the present invention will be described in more detail with reference to Examples. However, the following Examples are merely illustrative to aid understanding of the present invention, and the scope of the present invention is not limited thereto.

### Example 1-1: Synthesis of 5-bromo-3-cyano-1-isopropyl-indole

5-bromo-3-cyano-1H-indole (20 g, 90.5 mmol) was dissolved in 100 mL of acetone, then Cs₂CO₃ (50.1 g, 153.8 mmol) and 2-iodopropane (26.1 g, 153.8 mmol) were added, and the mixture was stirred under reflux for 2 hours. After completion of the reaction, the reaction solution was removed by distillation under reduced pressure, EtOAc (100 mL) was added, and washing with purified water was performed. The organic layer was separated, and the solvent was removed to obtain 23.5 g (98% yield) of the title compound.

¹H-NMR (CDCl₃) δ 7.90 (1H, d), 7.70 (1H, s), 7.43 (1H, dd), 7.32 (1H, d), 4.72-4.62 (1H, m), 1.57 (6H, d)

### Example 1-2: Synthesis of 1-(3-cyano-1-isopropyl-indol-5-yl)pyrazole-4-carboxylic acid ethyl ester

5-bromo-3-cyano-1-isopropyl-indole (23.5 g, 89.3 mmol) and 1H-pyrazole-4-carboxylic acid ethyl ester (12.5 g, 89.3 mmol) were added to 107 mL of toluene. CuI, 1,2-cyclohexanediamine, and K₂CO₃ were added, and the mixture was stirred under reflux for 2 days. The solvent was distilled off under reduced pressure, ethyl acetate (EtOAc) was added, washing with NH₄OH aqueous solution was performed, and the organic layer was filtered through Na₂SO₄/silica gel. The solvent was distilled off under reduced pressure, and crystallization with isopropyl alcohol (IPA) was performed to obtain 16.1 g (56% yield) of the title compound.

¹H-NMR (CDCl₃) δ 8.45 (1H, s), 8.13 (1H, s), 8.03 (1H, d), 7.80 (1H, s), 7.75 (1H, dd), 7.54 (1H, d), 4.79-4.69 (1H, m), 4.36 (2H, q), 1.61 (6H, d), 1.40 (3H, t)

### Example 1-3: Synthesis of 1-(3-cyano-1-isopropyl-indol-5-yl)pyrazole-4-carboxylic acid

1-(3-cyano-1-isopropyl-indol-5-yl)pyrazole-4-carboxylic acid ethyl ester (16 g, 49.6 mmol) was dissolved in a mixed solvent of 25 mL of tetrahydrofuran (THF) and 25 mL of methanol (MeOH), and then 25 mL of 10 N NaOH aqueous solution was added. The mixture was stirred at room temperature for 2 hours, and 25 mL of purified water was added. The solid produced by addition of c-HCl was filtered to obtain 12.9 g (88% yield) of the title compound.

¹H-NMR (DMSO-d₆) δ 12.56 (1H, br), 9.10 (1H, s), 8.53 (1H, s), 8.16 (1H, d), 8.06 (1H, s), 7.91-7.85 (2H, m), 4.92-4.86 (1H, m), 1.48 (6H, d)

1. A method for preparing a xanthine oxidase inhibitor of the following Chemical Formula 1, the method comprising the following steps:
i) preparing a compound of Chemical Formula 3 by introducing an R2 substituent into a compound of Chemical Formula 2,
ii) reacting the compound of Chemical Formula 3 with a compound of the following Chemical Formula 4 to prepare a compound of Chemical Formula 5, and
iii) preparing a compound of Chemical Formula 1 by removing an R4 substituent from the compound of Chemical Formula 5: 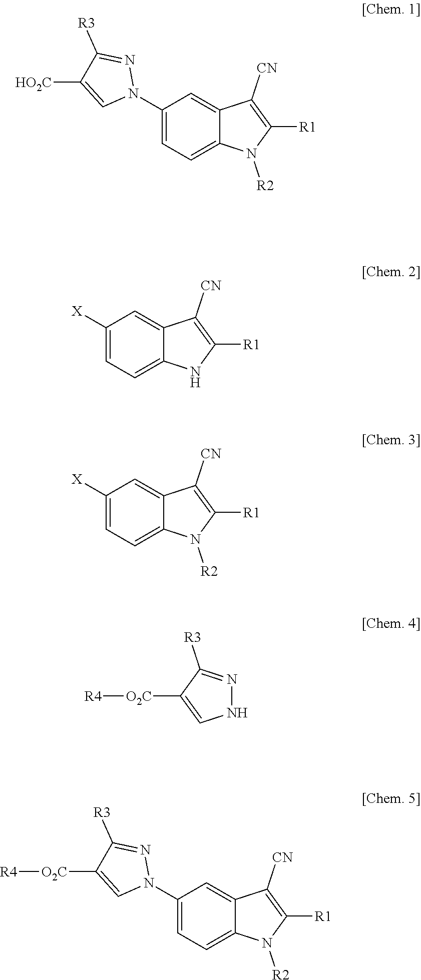
wherein,
R1 is hydrogen, halogen, C₁-C₇ alkyl, C₁-C₇ alkoxy-C₁-C₇ alkyl or phenyl;
R2 is hydrogen; C₁-C₇ alkyl unsubstituted or substituted with a substituent selected from halogen, C₃-C₇ cycloalkyl or O—R6, wherein R6 represents C₁-C₄ alkyl; C₃-C₇ cycloalkyl; or 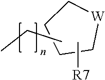
(wherein W represents O or S, R7 represents hydrogen or C₁-C₄ alkyl, and n is an integer 0 to 3);
R3 is hydrogen, halogen or C₁-C₇ alkyl;
R4 is C₁-C₇ alkyl or C₃-C₇ cycloalkyl; and
X is F, Cl, Br or I.

2. The preparation method according to claim 1, wherein R2 is unsubstituted C₁-C₇ alkyl.

3. The preparation method according to claim 2, wherein R2 is isopropyl.

4. The preparation method according to claim 3, wherein the compound of Chemical Formula 2 is reacted with 2-iodopropane and CS₂CO₃ in step (i).

5. The preparation method according to claim 1, wherein an organic solvent in step (ii) is one or more selected from toluene, xylene, dimethylformamide (DMF), or dimethyl sulfoxide (DMSO).

6. The preparation method according to claim 1, wherein a copper catalyst in step (ii) is one or more selected from CuI, Cu(OAc)₂, Cu, Cu₂O or CuO.

7. The preparation method according to claim 1, wherein a base in step (ii) is one or more selected from potassium carbonate, cesium carbonate, potassium phosphate tribasic, triethylamine or sodium tert-butoxide.

8. The preparation method according to claim 1, wherein a ligand in step (ii) is one or more selected from 1,2-cyclohexanediamine, N,N′-dimethyl-1,2-cyclohexanediamine, N,N′-dimethylethylenediamine, 1,10-phenanthroline, proline, an oxime ligand or a tetradentate ligand.

9. The preparation method according to claim 1, wherein step (iii) is carried out by hydrolyzing an ester with a base.

10. The preparation method according to claim 9, wherein the base is one or more selected from sodium hydroxide (NaOH), lithium hydroxide (LiOH), calcium hydroxide (Ca(OH)₂), or potassium hydroxide (KOH).
